(19) United States
(12) Patent Application Publication
(10) Pub. No.: US 2024/0197224 A1
(43) Pub. Date: Jun. 20, 2024

(54) AUTOMATED HAND-BIOMETRIC ANALYZER

(71) Applicants: Jimmy Youngblood, Richmond, TX (US); Matthew Currier, Cypress, TX (US)

(72) Inventors: Jimmy Youngblood, Richmond, TX (US); Matthew Currier, Cypress, TX (US)

(21) Appl. No.: 18/540,865

(22) Filed: Dec. 14, 2023

Related U.S. Application Data
(60) Provisional application No. 63/432,394, filed on Dec. 14, 2022.

(51) Int. Cl. A61B 5/22; A61B 5/00; A61B 5/0205; G06N 20/00

(52) U.S. Cl. CPC A61B 5/225; A61B 5/0026; A61B 5/0205; G06N 20/00

(57) ABSTRACT

An automated hand-biometric analyzer is provided. The analyzer comprises memory, a communication interface, a controller coupled to the memory, communication interface, and one or more load cell sensors configured to measure a hand grip strength of a user. A housing holds the memory, communication interface, controller, and load cell sensors, wherein the housing is configured to be received within the hand of a user. A gripping surface extends along a least a first portion of an outer surface of the housing and includes one or more connection points where the one or more connection points are communicatively coupled to the load cells. One or more cardiovascular sensors located on a second portion of the outer surface of the housing and configured to receive a palm of the hand of the user and measure cardiovascular parameters of the user.

### CLAIM OF PRIORITY

This application claims priority to and the benefit of U.S. Provisional Patent Application No. 63/432,394, titled AUTOMATED HAND-BIOMETRIC ANALYZER, and filed on Dec. 14, 2023, at the United States Patent and Trademark Office, the entire content of which is incorporated by reference herein as if fully set forth below in its entirety for all applicable purposes.

### TECHNICAL FIELD

The present disclosure generally relates to an automated hand-biometric analyzer. More specifically, a handheld device having multiple sensors which can measure biometrics generated by, or present at, the hand.

### BACKGROUND

There are many medical diagnostic tests that allow quick, proven, and highly researched generalizability specific to many health factors including those that have the largest impact on health and mortality. One of the most highly utilized tests is the handgrip strength test. Handgrip strength is considered a valuable indicator of overall health and has been associated with various health correlations including:
Mortality Risk: Research has shown that lower grip strength is associated with an increased risk of mortality, both in older and younger populations. It can be an indicator of frailty and general health status.
Cardiovascular Health: Grip strength has been correlated with cardiovascular health. Studies have shown associations between low grip strength and increased risk of cardiovascular events.
Diabetes Risk: There is evidence suggesting that low grip strength may be associated with an increased risk of type 2 diabetes.
Cognitive Function: Some studies suggest a correlation between grip strength and cognitive function. Lower grip strength has been linked to a higher risk of cognitive decline in older adults.
Bone Density: Some studies have explored the relationship between grip strength and bone density, suggesting that higher grip strength may be associated with greater bone density.
Muscle Mass and Function: Handgrip strength is often used as a proxy for overall muscle strength, and it can be indicative of an individual's muscle mass and function.
Inflammation: Chronic inflammation is associated with various health conditions. Grip strength has been inversely correlated with markers of inflammation in some studies.
Nutritional Status: Low grip strength may be associated with poor nutritional status, particularly in older adults.
Functional Ability: Handgrip strength is often used as a measure of functional ability, particularly in older adults. It can predict an individual's ability to perform activities of daily living.
Rehabilitation Progress: In rehabilitation settings, monitoring changes in grip strength can be useful for assessing progress and the effectiveness of interventions.

The use of grip strength measurements as a tool in medicine has a long history, and it has been employed in various forms for many decades. The grip strength test has been particularly prevalent in the fields of rehabilitation, geriatrics, and occupational medicine. While it's challenging to pinpoint an exact date of origin, some key points in the historical development of grip strength testing in medicine:
Early to Mid-20th Century: Grip strength measurements gained popularity in the mid-20th century as part of physical medicine and rehabilitation practices. The emphasis was often on assessing functional capacity and rehabilitation potential.
Geriatrics and Aging Research: In the latter half of the 20th century, grip strength became an important measure in geriatric medicine. Researchers and clinicians recognized its association with frailty, functional decline, and overall health in older adults.
Occupational Health: Grip strength tests have been used in occupational health settings to assess an individual's ability to perform specific job tasks and to identify potential risks for musculoskeletal injuries.
Research and Standardization: Over time, researchers and health professionals worked to standardize the grip strength test and establish normative data for different age groups and populations. This standardization has facilitated the comparison of grip strength results across studies and clinical settings.
Connection to Chronic Diseases: Grip strength measurements have been incorporated into studies investigating associations with chronic diseases such as cardiovascular disease, diabetes, and osteoporosis.
Recent Advances: In recent years, with advancements in technology, digital dynamometers have been developed for more precise and objective grip strength assessments.

Currently, the standard procedure for grip strength testing in medicine is as follows:
1. Introduction: Explain the purpose of the test to the individual. Ensure the individual is comfortable and understands the procedure.
2. Positioning: The individual should be seated with their feet flat on the floor and their elbow flexed at 90 degrees. The forearm should be in a neutral position (not pronated or supinated).
3. Dynamometer Calibration: Calibrate the handheld dynamometer according to the manufacturer's instructions.
4. Testing: Instruct the individual to grip the dynamometer with maximum force when prompted. The dynamometer should be held in the dominant hand unless there's a specific reason to test the non-dominant hand. Perform the test three times for each hand, with a short rest between trials.
5. Recording: Record the maximum force (in kilograms or pounds) exerted during each trial. Calculate the average grip strength for each hand.
6. Documentation: Document the individual's demographic information, hand dominance, and any relevant health history. Note any pain or discomfort experienced during the test.
7. Interpretation: Compare the individual's grip strength to established norms for their age and gender. Consider any relevant factors, such as hand dominance and overall health.

Unfortunately, there has been very limited innovation in the technology which severely limits the ability to both diagnose and gather data. Problems with existing grip strength testing in medicine include:
Static Measurement: The grip strength test typically provides a static measurement at a single point in time. Dynamic assessments that evaluate muscle function during movement may offer additional insights into functional capacity.
Tool and Protocol Variability: The choice of dynamometer and the testing protocol can vary, potentially leading to differences in results. Standardization of equipment and protocols is crucial for consistency across studies and clinical settings.
Lack of Sensitivity to Early Changes: Grip strength may not be sensitive enough to detect subtle changes in muscle function, particularly in the early stages of certain conditions. More sensitive measures may be required for early detection of muscle weakness.
Limited Specificity: Grip strength is a general measure of upper limb strength but may not provide specific information about the strength of individual muscle groups. It may not be the ideal tool for diagnosing specific localized muscle issues.
Influence of Factors Beyond Muscle Strength: Grip strength can be influenced by factors other than muscle strength, such as joint health, pain, and psychological factors. Therefore, it should be used in conjunction with other assessments for a comprehensive evaluation.
Population Differences: Normative data for grip strength can vary based on factors such as age, gender, and population. It's essential to consider these differences when interpreting results, and more research is needed to establish standardized norms for diverse populations.
Not a Comprehensive Health Assessment: While grip strength is associated with various health outcomes, it is just one component of a comprehensive health assessment. Other factors, such as cardiovascular fitness, flexibility, and balance, should also be considered for a holistic evaluation.
Limited Application in Certain Conditions: In some medical conditions, such as neurological disorders or conditions affecting the lower extremities, grip strength may have limited relevance. In these cases, other assessments specific to the condition may be more appropriate.

One example of an existing device that can measure biometrics of an individual is a force dynamometer. Force dynamometers are used for measuring the back, grip, arm, and/or leg strength of individuals (such as, athletes, patients, and workers) to evaluate physical status, performance, and task demands. Typically, the force applied to a lever or through a cable is measured and then converted to a moment of force by multiplying by the perpendicular distance from the force to the axis of the level.

Additionally, handgrip dynamometers may be used for measuring the maximum isometric contraction strength of the hand and forearm muscles. Handgrip dynamometers are used for testing handgrip strength of patients suffering from conditions that impair hand strength and for tracking improvements during rehabilitation. They are also used by athletes involved in strength training or participating in sports in which the hands are used for catching, throwing, or lifting such as gymnasts, tennis players, and rock climbers.

However, these existing handheld dynamometers typically measure only maximum force output of the hand and report it using manual methods or closed protocols and data via a proprietary application.

In addition to dynamometers, various devices exist that can measure biometrics generated using a hand or a portion of a hand. For example, a pulse oximeter can measure the oxygen level (oxygen saturation) of the blood though a finger of the user. Pulse oximeters typically provide the data on the device or via a proprietary application. Furthermore, devices exist that measure bioimpedance which is a measure of how well the body impedes electric current flow, such as, two handheld devices with a wired electrical connection between them to measure bioimpedance across the body.

While various devices currently exist that can measure biometrics of a user, not a single device currently exists which is a platform for measuring a variety of hand-biometrics at once; that is, any sensor feedback which can be measured at the hand. Nor do they measure and evaluate feedback dynamically. What is needed in the art is a handheld device that has multiple sensors which can measure biometrics generated by, or present at, the hand and individual fingers and which can aggregate and analyze this data to provide a more wholistic health evaluation per hand or between both hands. Furthermore, what is needed is a platform that also supports integration of multiple devices (one in each hand) and a central computing device (local or remote) and can provide data through an open system to allow 3rd party software integrations as well as a system that can provide analysis of that feedback over time for discerning health outcomes.

### SUMMARY

The following presents a simplified summary of one or more aspects of the present disclosure to provide a basic understanding of such aspects. This summary is not an extensive overview of all contemplated features of the disclosure. It is intended neither to identify key or critical elements of all aspects of the disclosure nor to delineate the scope of any or all aspects of the disclosure. Its sole purpose is to present some concepts of one or more aspects of the disclosure in a simplified form as a prelude to the more detailed description presented below.

In one example, an automated hand-biometric analyzer is provided. The analyzer comprises a memory; a communication interface; a controller coupled to the memory and the communication interface; one or more load cell sensors communicatively coupled to the controller and configured to measure a hand grip strength of a user; a housing that holds the memory, the communication interface, the controller, and the one or more load cell sensors, wherein the housing is a configured to be received within a hand of a user; a gripping surface extending along a least a first portion of an outer surface of the housing, the gripping surface having one or more connection points where the one or more connection points are communicatively coupled to the one or more load cells; and one or more cardiovascular sensors located on a second portion of the outer surface of the housing and configured to receive a palm of the hand of the user and measure cardiovascular parameters of the user.

According to one aspect, the analyzer further comprises one or more load cell amplifiers communicatively coupled to the one or more load sensors for increasing a strength of signals coming from the one or more load sensors.

According to another aspect, the gripping surface is comprised of two or more separate individual gripping surfaces, where each of the individual gripping surfaces is configured to receive a separate finger from the hand of the user to measure a finger grip strength individually or a combination of fingers.

According to yet another aspect, the one or more connection points are configured to contact one or more fingers of the hand of the user for measuring conductivity or impedance between the one or more load cell sensors or the one or more cardiovascular sensors.

According to yet another aspect, the one or more connection points are configured to contact the fingers or the palm of the user for measuring conductivity or impedance between one or more second analyzer sensors located on a second hand-biometric analyzer communicatively coupled to the automatic hand-biometric analyzer.

According to yet another aspect, the analyzer further comprises a communication port communicatively coupled to the controller for communicating through a wired connection to an electronic device.

According to yet another aspect, the electronic device is at least one of a second hand-biometric analyzer, a local computing device, and a remote computing device.

According to yet another aspect, wherein the controller communicates wirelessly with an electronic device.

According to yet another aspect, the electronic device is at least one of a second hand-biometric analyzer, a local computing device, and a remote computing device.

In another example, an automated hand-biometric analyzer is provided. The analyzer includes a memory; a communication interface; and a controller coupled to the memory and the communication interface. The controller is configured to collect a first set of biometric data from a user using one or more load cell sensors communicatively coupled to the controller and configured to measure a hand grip strength of the user; collect a second set of biometric data from the user from one or more cardiovascular sensors coupled to the controller and configured to measure cardiovascular parameters of the user; analyzing the first and second sets of biometric data; and providing feedback on the first and second sets of biometric data collected.

According to one aspect, wherein the controller is configured to store the first and second sets of biometric data on the analyzer.

According to another aspect, the controller is configured to store the first and second sets of biometric data on a local computing device.

According to yet another aspect, the controller is configured to store the first and second sets of biometric data on a remote computing device.

According to yet another aspect, the controller is configured to store the first and second sets of biometric data on a second hand-biometric analyzer.

According to yet another aspect, the feedback is provided in real-time to one or more users.

According to yet another aspect, the first and second sets of biometric data are used to generate metadata, and where the metadata is reported through statistical analysis.

According to yet another aspect, the first and second sets of biometric data are used to generate or train machine-learning models or use machine-learning for reporting or analysis.

### BRIEF DESCRIPTION OF THE DRAWINGS

The features, nature, and advantages of the present aspects may become more apparent from the detailed description set forth below when taken in conjunction with the drawings in which like reference characters identify correspondingly throughout.

FIG. 1 illustrates a side plan view of an automated hand-biometric analyzer, according to one example of the present disclosure.

FIG. 2 illustrates a front plan view of the automated hand-biometric analyzer of FIG. 1.

FIG. 3 illustrates an example visualization displaying the real-time grip strength of the right and left hands [alternating] of a user.

FIG. 4 illustrates example feedback presented to the user after completion of the handgrip strength test.

FIG. 5 is a block diagram of an automated hand-biometric analyzer, according to some aspects of the disclosure.

FIG. 6 is a block diagram illustrating an example of a hardware implementation of an automated hand-biometric analyzer employing a processing system, according to some aspects of the disclosure.

### DETAILED DESCRIPTION

In the following description, specific details are given to provide a thorough understanding of the described implementations. However, it will be understood by one of ordinary skill in the art that the implementations may be practiced without these specific details. For example, certain aspects may be illustrated with simplified representations in order not to obscure the implementations in unnecessary detail. In other instances, well-known techniques may be shown in broad block form in order not to obscure the described implementations.

### Handheld Device—Automated Hand-Biometric Analyzer

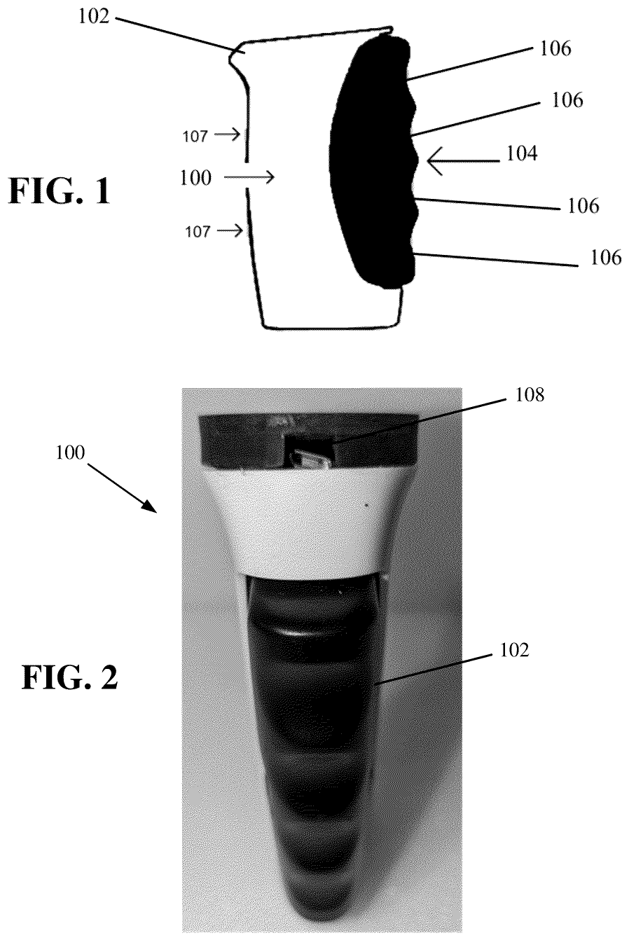

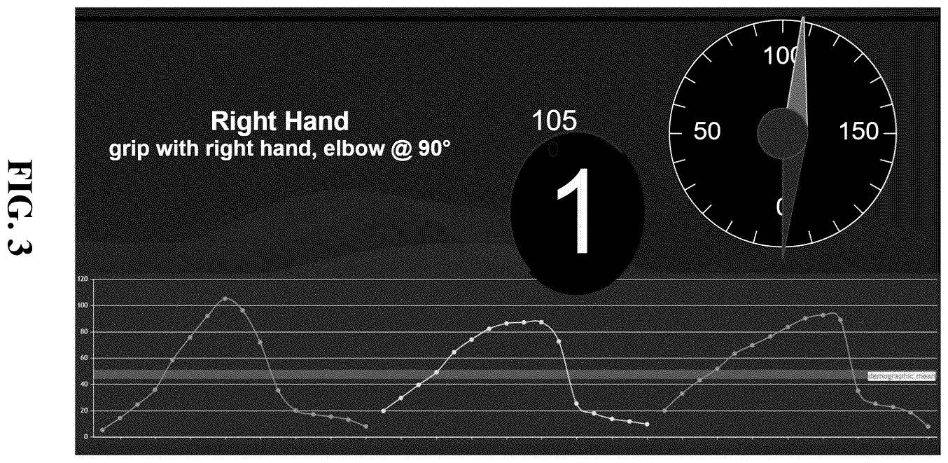

FIG. 1 illustrates a side plan view of an automated hand-biometric analyzer 100, according to one example of the present disclosure. FIG. 2 illustrates a front plan view of the automated hand-biometric analyzer 100 of FIG. 1. As shown, the automated hand-biometric analyzer 100 is a single handheld unit or device having a housing 102 configured to be received within a hand of a user. A gripping surface 104 extends at least partially along a length of a first portion of an outer surface of the housing 102 and is adapted to receive at least one finger of the user. The gripping surface 104 is attached to one or more force sensors (or load cell censors) located internally within the housing 102 for measuring the force output of the entire hand or individual fingers. Conductive points or sensors 106 (hereinafter used interchangeably), located on the gripping surface, may be used to measure resistivity or impedance between any two, or more conductive points 106, or between similar conductive points on a second hand-biometric analyzer or other similar device. Although four (4) conductive points 106 are shown, this is by way of example only and there may be more than four (4) or less than four (4) conductive points. Additionally, these conductive points 106 may be placed anywhere on the outer surface 102 of the device 100 that may come into contact with the hand. The conductive points or sensors 106 collect a first set of biometric data.

According to one aspect, one or more cardiovascular sensors (such as photoplethysmographic or temperature sensors) 107, which can be used to measure pulse oximetry, heart rate, blood pressure, cardiac output, stroke volume, various blood tests or other cardiovascular metrics related to the heart and circulatory system, may be located on a second portion of the outer surface of the analyzer 100, where the second portion is different than the first portion. As shown in FIG. 1, the one or more cardiovascular sensors 107 are located on the outer surface such that when a user holds the analyzer 100 in his or her hands, the user's palms come into contact with the sensors 107. However, the sensors 107 may be placed anywhere on the outer surface of the analyzer 100 that may come into contact with the hand of a user. Although two (2) sensors are shown, this is by way of example only and there may be more than two (2) sensors or less than two (2) sensors. The cardiovascular sensors 107 collect a second set of biometric data.

The first and second sets of biometric data are used to generate metadata, where the metadata is reported through statistical analysis. The first and second sets of biometric data may also be used to generate or train machine-learning models or use machine-learning for reporting or analysis.

The first and second set of biometric data may be stored on a local computing device, a remote computing device, a second hand biometric analyzer, and/or any other type of electronic device that is capable of communicating with the automated hand biometric analyzer.

According to one embodiment, the handheld automated hand-biometric analyzer 100 having multiple sensors can measure biometrics generated by, or present at, the hand. The automated hand-biometric analyzer 100 may be held in a single hand of the user. Measurements that may be obtained by the automated hand-biometric analyzer 100 include, but are not limited to, grip force production (of the entire hand and individual fingers), heart rate, blood oximetry, blood-pressure, bio-impedance (between various locations on the hand or (via an interface cable) between points on this hand and another location on the body), and temperature (at one or more points.) This includes sensor locations in a second, identical device held in the opposite hand. These measurements can be stored and displayed locally or interfaced with a local or remote computer application.

The automated hand-biometric analyzer 100 of the present disclosure can provide medically accepted feedback on the various measurements obtained. The feedback may be provided to the user or anyone else authorized by the user in real-time or may be stored for later retrieval. The series of biometric measures obtained using the automated hand-biometric analyzer 100 may be compared to nominal data, via direct comparison or algorithm or machine learning model, as well as the user's historical data to account for variability and limited specificity of testing procedures. The nominal data may be stored in memory on the analyzer 100 or may be stored in local or remote computers. Higher data acquisition rates per user, and for the system overall, yields more data which makes statistical analysis more efficacious and allows for better training of machine learning models both for individual users and for the system as a whole.

The automated hand-biometric analyzer 100 of the present disclosure is capable of both wired and wireless communication with local or remote computers for the purpose of data transfer. The automated hand-biometric analyzer 100 may include one or more ports, such as a USB port 108, for connecting to computers or other devices. For example, the automated hand-biometric analyzer 100 is capable of connecting (either wired or wirelessly) to other devices, such as another analyzer, to measure bio-impedance across the body of the user. A wired connection, such as a USB or serial cable may be used to connect the controller of the analyzer to another electronic device such as a computer, server, or another analyzer. Alternatively, the controller may communicate electronically with another electronic device through a wireless connection such as WiFi or Bluetooth.

Reporting of measurements made by the automated hand-biometric analyzer 100 can be accomplished on the analyzer 100 itself or via a local or remote computer application. These reports can be delivered to the user of the analyzer, the owner of the analyzer (if different), or an authorized third-party (such as a practitioner provided healthful analysis and feedback.)

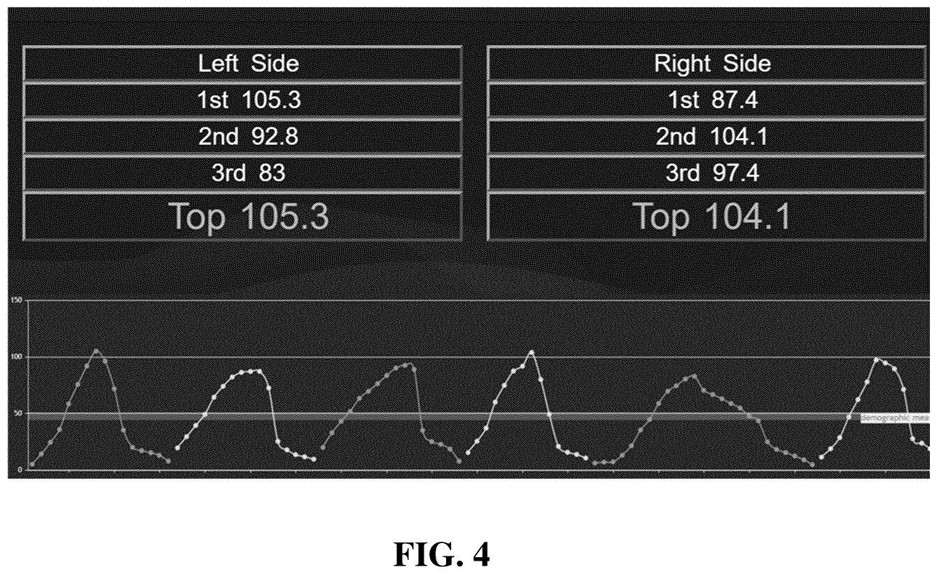

In one implementation, the automated hand-biometric analyzer 100 may be used to measure maximal grip strength of each hand three times in accordance with basic, commonly accepted medical standards. FIG. 3 illustrates an example visualization displaying the real-time grip strength of the right and left hands [alternating] of a user. As shown, the grip strength of the right hand is measured at 105 pounds at that moment. FIG. 4 illustrates example feedback presented to the user after completion of the handgrip strength test. The includes a trend chart showing the output per time interval for each hand tested 3 times. The trend chart may identify the separate hands (i.e. left hand and right hand) by coloring coding (e.g. green/left, yellow/right) or any other identifier such as a unique line pattern.

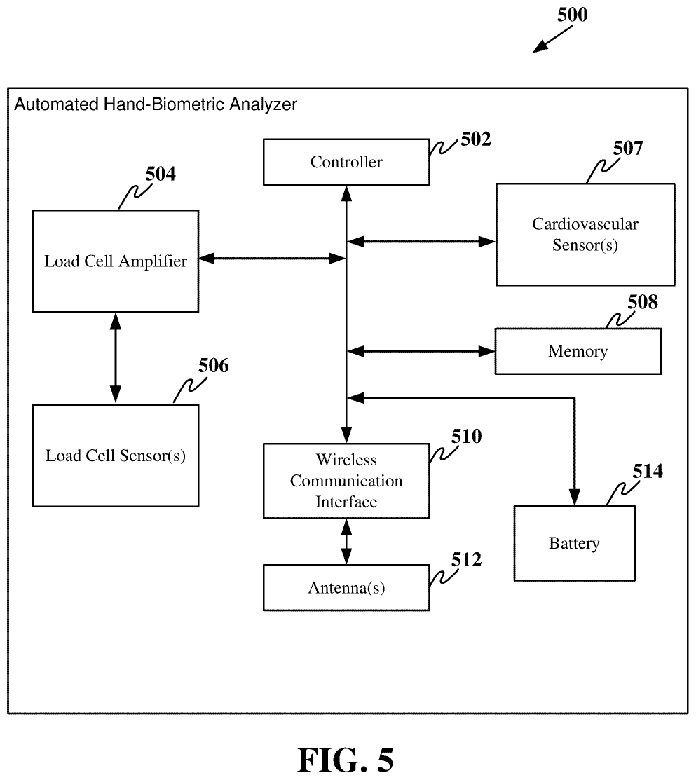

Turning to FIG. 5, a block diagram of an automated hand-biometric analyzer 500 according to some aspects of the disclosure is provided. The automated hand-biometric analyzer 500 may include a controller 502 (e.g., a processing system), a load cell amplifier 504, one or more load cell sensors 506, and one or more cardiovascular sensors (such as photoplethysmographic and/or temperature sensors) 507. A load cell converts a force such as tension, compression, pressure, or torque into an electrical signal that can be measured and standardized. It is a force transducer. As the force applied to the load cell increases, the electrical signal changes proportionally. The load cell amplifier 504 is a device that can increase the strength of signals coming from a load cell. Sometimes, the signals produced by the load cell can be feeble and low strength signals may not work with certain components of the measuring system like a data logger for load cells or load meter. Although a single load cell sensor and load cell amplifier are shown, multiple load cell sensors and load cell amplifiers may be utilized to obtain different measures from different parts of the hand of a user. The one or more cardiovascular sensors 507 (such as photoplethysmographic and/or temperature sensors) can be used to measure pulse oximetry, heart rate or other cardiovascular metrics of the user.

The automated hand-biometric analyzer 500 may receive power from a power source, such as a battery 514, a capacitor, or any other commonly available electricity sources. The automated hand-biometric analyzer 500 may also include a wireless communication interface 510 and one or more antennas 512. The wireless communication interface 510 may be a wireless transceiver. For example, the wireless communication interface 510 may be configured to permit a user to interface with the automated hand-biometric analyzer 500 via Bluetooth®, WiFi®, WiMAX®, LTE, 4G, 5G and beyond, or the like. The one or more antennas 512 may be configured to operate at the frequency or frequencies utilized by the wireless communication interface 510.

According to some aspects, the controller 502 of the automated hand-biometric analyzer 500 may provide for remote or local control of the automated hand-biometric analyzer 500. The controller 502 may accept and may store (e.g., in a memory 508) the data measured by the sensors. The controller 502 may control the automated hand-biometric analyzer 500 and may also control one or more other automated hand-biometric analyzers that may be similar to or different from the automated hand-biometric analyzer 500.

According to one aspect of the disclosure, as described above, the automated hand-biometric analyzer 500 may include a grip portion extending along a longitudinal axis for gripping by at least a portion of a human hand, and a sensor or plurality of sensors distributed along or below a surface of the grip portion. The sensor or plurality of sensors detect force exerted by the portion of the human hand in directions radial and tangential to the grip portion such that grip strength and wrist strength of the human may be sensed by the sensor or plurality of sensors.

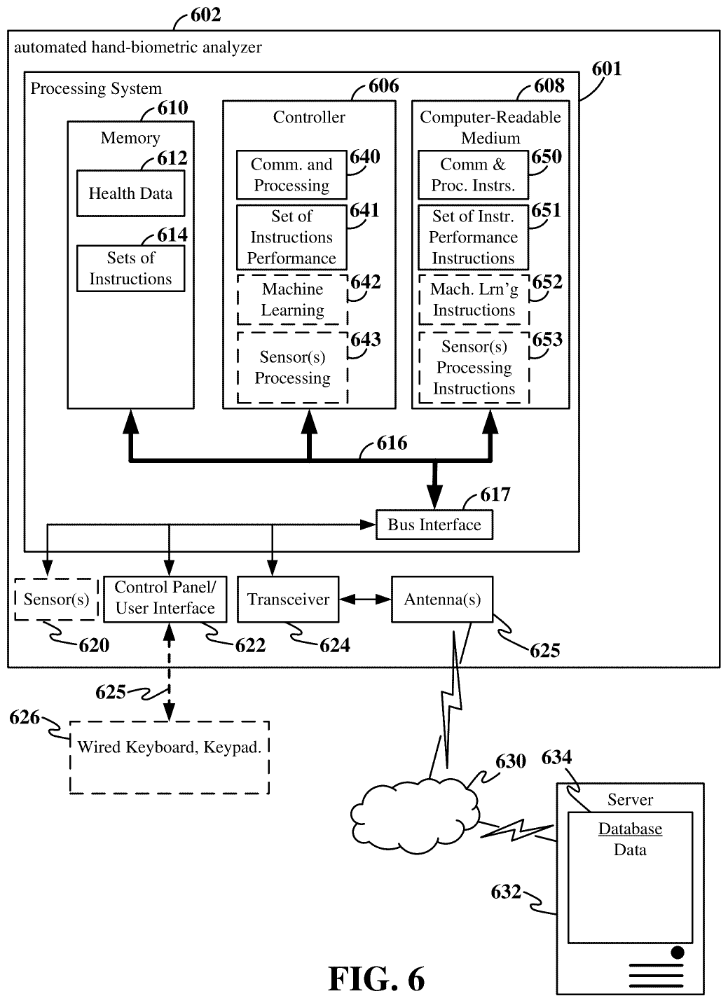

FIG. 6 is a block diagram illustrating an example of a hardware implementation of an automated hand-biometric analyzer 600 employing a processing system, according to some aspects of the disclosure. For example, the automated hand-biometric analyzer 600 may correspond to the automated hand-biometric analyzer 500 as shown and described in connection with FIG. 5 as well as the automated hand-biometric analyzer 100 as shown and described in connection with FIGS. 1-2.

In accordance with various aspects of the disclosure, an element, or any portion of an element, or any combination of elements, may be implemented with a processing system 601 that includes one or more controllers (or processors), such as controller 606. Examples of controller 606 include microprocessors, microcontrollers, digital signal processors (DSPs), field programmable gate arrays (FPGAs), programmable logic devices (PLDs), state machines, gated logic, discrete hardware circuits, and other suitable hardware configured to perform the various functionality described throughout this disclosure. In various examples, the automated hand-biometric analyzer 600 may be configured to perform any one or more of the functions described herein.

In this example, the processing system 601 may be implemented with a bus architecture, generally represented by the bus 616. The bus 616 may include any number of interconnecting buses and bridges depending on the specific application of the processing system 601 and the overall design constraints. The bus 616 links together various circuits, including one or more controllers (represented generally by the controller 606), a memory 610, and computer-readable media (represented generally by the computer-readable medium 608). The bus 616 may also link various other circuits such as timing sources, peripherals, voltage regulators, and power management circuits, which are well known in the art and will not be described further.

A bus interface 617 provides an interface between the bus 616 and a plurality of additional circuits/functions, including sensor(s) (or connection points) 620, a control panel/user interface 622, a transceiver 624, and antenna(s) 625. The preceding lists were exemplary and non-limiting. The sensors 620 may include load cell sensors and cardiovascular sensors (such as photoplethysmographic and/or temperature sensors), as described above.

The transceiver 624 and antenna(s) 625 may provide a communication interface or a means for communicating with various other apparatus over a transmission medium (e.g., an air interface). The control panel/user interface 622 may provide a user with a way to directly interface with the automated hand-biometric analyzer 600. The control panel/user interface 622 may be operationally coupled to a wired keyboard or a keypad (any of which may include a remote display, a touch screen, a speaker, a microphone, etc.), which may provide optional ways for a user to interface with the automated hand-biometric analyzer 600 according to some aspects described herein. The wired keyboard or keypad may be operationally coupled to the control panel/user interface 622 by a cable 625.

The controller 606 may be responsible for managing the bus 616 and general processing, including executing software stored on the computer-readable medium 608. The software, when executed by the controller 606, causes the processing system 601 to perform the various functions for measuring a variety of hand-biometrics generated by, or present at, the hand and which can aggregate and analyze this data to provide a more wholistic health evaluation.

The computer-readable medium 608 and the memory 610 may also be used for storing data manipulated by the controller 606 when executing software. For example, the memory 610 may store health data 612 and sets of instructions 614 generated measure the variety of hand-biometrics.

One or more controllers (such as the controller 606) in the processing system 601 may execute software. Software shall be construed broadly to mean instructions, instruction sets, code, code segments, program code, programs, subprograms, software modules, applications, software applications, software packages, routines, subroutines, objects, executables, threads of execution, procedures, functions, etc., whether referred to as software, firmware, middleware, microcode, hardware description language, or otherwise. The software may reside on the computer-readable medium 608.

The computer-readable medium 608 may be a non-transitory computer-readable medium. A non-transitory computer-readable medium includes, by way of example, a magnetic storage device (e.g., hard disk, floppy disk, magnetic strip), an optical disk (e.g., a compact disc (CD), or a digital versatile disc (DVD)), a smart card, a flash memory device (e.g., a card, a stick, or a key drive), a random access memory (RAM), a read-only memory (ROM), a programmable ROM (PROM), an erasable PROM (EPROM), an electrically erasable PROM (EEPROM), a register, a removable disk, and any other suitable medium for storing software and/or instructions that may be accessed and read by a computer. The computer-readable medium 608 may reside in the processing system 601, external to the processing system 601, or distributed across multiple entities, including the processing system 601. The computer-readable medium 608 may be embodied in a computer program product. By way of example, a computer program product may include a computer-readable medium in packaging materials. In some examples, the computer-readable medium 608 may be part of the memory 610. Those skilled in the art will recognize how best to implement the described functionality presented throughout this disclosure depending on the particular application and the overall design constraints imposed on the overall system. In some examples, the computer-readable medium 608 may be implemented on an article of manufacture, which may further include one or more other elements or circuits, such as the controller 606 and/or memory 610.

In some aspects, the automated hand-biometric analyzer 600 may include the memory 610 and the controller 606 coupled to the memory 610, the controller 606 and the memory 610, at least, may be configured to perform any of the methods, functions, or algorithms described herein. In some aspects of the disclosure, the controller 606 may include circuitry configured for various functions. For example, the controller 606 may include communication and processing circuitry/function 640 (also referred to as the communication and processing circuitry 640 for the sake of brevity), configured to communicate with other devices internal and external to the automated hand-biometric analyzer 600, for example, via interfaces therebetween.

In some examples, the communication and processing circuitry 640 may include one or more hardware components that provide the physical structure that performs processes related to communication (e.g., signal reception and/or signal transmission) and signal processing (e.g., processing a received signal and/or processing a signal for transmission). For example, the communication and processing circuitry 640 may include one or more modems. In some examples, the communication and processing circuitry 640 may include one or more hardware components that provide the physical structure that performs processes related to processing, such as, for example, obtaining health data 612 and sets of instructions 614 from the memory 610. In some implementations where the communication involves receiving data from sensor(s) 620, for example, the communication and processing circuitry 640 may obtain the data, process the data, and output the processed data. For example, the communication and processing circuitry 640 may output the processed data to another component of the controller 606, the memory 610, or the bus interface 617. In some examples, the communication and processing circuitry 640 may receive one or more of signals, messages, other information, or any combination thereof. In some examples, the communication and processing circuitry 640 may include functionality for a means for receiving and/or a means for transmitting.

In some aspects of the disclosure, the controller 606 may include a set of instructions performance circuitry/function 641 configured to obtain various measurements, such as grip force production (of the entire hand and individual fingers), heart rate, blood oximetry, blood-pressure, bio-impedance (between various locations on the hand or (via an interface cable) between points on this hand and another location on the body), and temperature (at one or more points.) For example, the set of instructions performance circuitry/function 641 may be utilized to interface with the sensors 620.

The set of instructions performance circuitry 641 may further be configured to execute set of instructions performance instructions 651 (e.g., software) stored on the computer-readable medium 608 to implement one or more functions described herein.

In some aspects of the disclosure, the controller 606 may include a machine learning circuitry/function 642 configured for various functions, including, for example, learning and adapting without following explicit instructions, by using algorithms and statistical models to analyze and draw inferences from patterns in data. The data may be received from the sensor(s) 620, for example, or received from the communication and processing circuitry/function 640 described above. The machine learning circuitry/function 642 may further be configured to execute machine learning instructions 652 (e.g., software) stored on the computer-readable medium 608 to implement one or more functions described herein.

In some aspects of the disclosure, the controller 606 may include, for example, sensor(s) processing circuitry/function 643, configured for various functions including, for example, obtaining information from sensor(s) 620 and acting on such information. For example, sensor(s) 620 may include a temperature sensor (e.g., a thermometer or a thermocouple), a grip force sensor, a heart rate sensor, a blood oximetry sensor, and a blood-pressure. The preceding list is exemplary and not limiting; other sensor(s) are within the scope of the disclosure. The sensor(s) processing circuitry/function 643 may further be configured to execute sensor(s) processing instructions 653 to implement one or more functions described herein.

According to some aspects, the automated hand-biometric analyzer 600 (or the controller 606) described herein may communicatively couple to a server 632, which may be a remote server and may pull data from and push data to a database 634 maintained at the server 632. Coupling may be via a communications network, represented by a cloud 630 in FIG. 5. Use of data stored in the database 634 at the server 632 may be aggregated and analyzed to provide a wholistic health evaluation of the user. Data storage on a server may also facilitate gathering data pertinent to individual users or groups of users sharing a common demographic. The gathered data may be used to build individual (personal) or group profiles and set individual (personal) or group goals. Data storage on the server 632 may also facilitate the aggregation of feedback comparing actual outcomes to the goals of users. A cloud computing system, for example, a system that utilizes the server 632, may enable broad storage of data. Additionally, further machine learning might be performed by the cloud computing system using all available data to glean fine-tune device measurement, feedback, and application for users and associated practitioners.

While the foregoing disclosure shows illustrative aspects, it should be noted that various changes and modifications could be made herein without departing from the scope of the disclosure as defined by the appended claims. The functions, steps and/or actions of the method claims in accordance with the aspects described herein need not be performed in any particular order. Furthermore, although elements of aspects disclosed herein may be described or claimed in the singular, the plural is contemplated unless limitation to the singular is explicitly stated.

Aspects described in connection with a given description, illustration, representation, or method may be substituted for aspects described in a different description, illustration, representation, or method.

The word “aspects” does not require that all aspects of the disclosure include the discussed tab, advantage, or mode of operation.

The word “coupled” is used herein to refer to the direct or indirect coupling between two objects. For example, if object A physically touches object B, and object B touches object C, then objects A and C may still be considered coupled to one another-even if they do not directly physically touch each other.

In one or more exemplary embodiments, the functions described may be implemented in hardware, software, firmware, or any combination thereof. If implemented in software as a computer program product, the functions may be stored on or transmitted over as one or more instructions or code on a computer-readable medium. Computer-readable media includes both non-transitory computer-readable storage media and communication media including any medium that facilitates transfer of a computer program from one place to another. A storage media may be any available media that can be accessed by a computer. By way of example, and not limitation, such computer-readable media can comprise RAM, ROM, EEPROM, CD-ROM or other optical disk storage, magnetic disk storage or other magnetic storage devices, or any other medium that can be used to carry or store desired program code in the form of instructions or data structures and that can be accessed by a computer. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, includes compact disc (CD), laser disc, optical disc, digital versatile disc (DVD), floppy disk and blu-ray disc where disks usually reproduce data magnetically, while discs reproduce data optically with lasers. Combinations of the above should also be included within the scope of computer-readable media.

The previous description of the disclosed embodiments is provided to enable any person skilled in the art to make or use the aspects described herein. Various modifications to these embodiments will be readily apparent to those skilled in the art, and the generic principles defined herein may be applied to other embodiments without departing from the spirit or scope of the invention. Thus, the disclosure is not intended to be limited to the embodiments shown herein but is to be accorded the widest scope consistent with the principles and novel aspects disclosed herein.

Although the present disclosure and its advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

As may be used herein, the term “operable to” or “configurable to” indicates that an element includes one or more of components, dimensions, circuits, instructions, modules, data, input(s), output(s), etc., to perform one or more of the described or necessary corresponding functions and may further include inferred coupling to one or more other items to perform the described or necessary corresponding functions. As may also be used herein, the term(s) “coupled,” “coupled to,” “connected to” and/or “connecting” or “interconnecting” includes direct connection or link between components or between nodes/devices and/or indirect connection between components or nodes/devices via an intervening item. As may further be used herein, inferred connections (i.e., where one element is connected to another element by inference) includes direct and indirect connection between two items in the same manner as “connected to.” As may be used herein, the terms “substantially” and “approximately” provides an industry-accepted tolerance for its corresponding term and/or relativity between items.

As used herein, the terms “comprise,” “comprises,” “comprising,” “having,” “including,” “includes” or any variation thereof, are intended to reference a nonexclusive inclusion, such that a process, method, article, composition or apparatus that comprises a list of elements does not include only those elements recited, but may also include other elements not expressly listed or inherent to such process, method, article, composition, or apparatus. Other combinations and/or modifications of the above-described structures, arrangements, applications, proportions, elements, materials, or components used in the practice of the present invention, in addition to those not specifically recited, may be varied or otherwise particularly adapted to specific environments, manufacturing specifications, design parameters, or other operating requirements without departing from the general principles of the same.

Moreover, reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is intended to be construed under the provisions of 35 U.S.C. § 112(f) as a “means-plus-function” type element, unless the element is expressly recited using the phrase “means for” or, in the case of a method claim, the element is recited using the phrase “step for.”

1. An automated hand-biometric analyzer comprising:
a memory;
a communication interface;
a controller coupled to the memory and the communication interface;
one or more load cell sensors communicatively coupled to the controller and configured to measure a hand grip strength of a user;
a housing that holds the memory, the communication interface, the controller, and the one or more load cell sensors, wherein the housing is a configured to be received within a hand of a user;
a gripping surface extending along a least a first portion of an outer surface of the housing, the gripping surface having one or more connection points where the one or more connection points are communicatively coupled to the one or more load cells; and
one or more cardiovascular sensors located on a second portion of the outer surface of the housing and configured to receive a palm of the hand of the user and measure cardiovascular parameters of the user.

2. The analyzer of claim 1, further comprising:
one or more load cell amplifiers communicatively coupled to the one or more load sensors for increasing a strength of signals coming from the one or more load sensors.

3. The analyzer of claim 1, wherein the gripping surface is comprised of two or more separate individual gripping surfaces, where each of the individual gripping surfaces is configured to receive a separate finger from the hand of the user to measure a finger grip strength individually or a combination of fingers.

4. The analyzer of claim 1, wherein the one or more connection points are configured to contact one or more fingers of the hand of the user for measuring conductivity or impedance between the one or more load cell sensors or the one or more cardiovascular sensors.

5. The analyzer of claim 1, wherein the one or more connection points are configured to contact the fingers or the palm of the user for measuring conductivity or impedance between one or more second analyzer sensors located on a second hand-biometric analyzer communicatively coupled to the automatic hand-biometric analyzer.

6. The analyzer of claim 1, further comprising a communication port communicatively coupled to the controller for communicating through a wired connection to an electronic device.

7. The analyzer of claim 6, wherein the electronic device is at least one of a second hand-biometric analyzer, a local computing device, and a remote computing device.

8. The analyzer of claim 1, wherein the controller communicates wirelessly with an electronic device.

8. The analyzer of claim 8, wherein the electronic device is at least one of a second hand-biometric analyzer, a local computing device, and a remote computing device.

9. An automated hand-biometric analyzer comprising:
a memory;
a communication interface; and
a controller coupled to the memory and the communication interface, wherein the controller is configured to:
collect a first set of biometric data from a user using one or more load cell sensors communicatively coupled to the controller and configured to measure a hand grip strength of the user;
collect a second set of biometric data from the user from one or more cardiovascular sensors coupled to the controller and configured to measure cardiovascular parameters of the user;
analyzing the first and second sets of biometric data; and
providing feedback on the first and second sets of biometric data collected.

10. The analyzer of claim 9, wherein the controller is configured to store the first and second sets of biometric data on the analyzer.

11. The analyzer of claim 10, wherein the controller is configured to store the first and second sets of biometric data on a local computing device.

12. The analyzer of claim 9, wherein the controller is configured to store the first and second sets of biometric data on a remote computing device.

13. The analyzer of claim 9, wherein the controller is configured to store the first and second sets of biometric data on a second hand-biometric analyzer.

14. The analyzer of claim 9, wherein the feedback is provided in real-time to one or more users.

15. The analyzer of claim 9, wherein the first and second sets of biometric data are used to generate metadata, and where the metadata is reported through statistical analysis.

16. The analyzer of claim 9, wherein the first and second sets of biometric data are used to generate or train machine-learning models or use machine-learning for reporting or analysis.
